(19) United States
(12) Patent Application Publication
(10) Pub. No.: US 2006/0178651 A1
(43) Pub. Date: Aug. 10, 2006

(54) Unitary stretch panel and method of manufacture

(76) Inventor: Frank S. Glaug, Chester Springs, PA (US)

Correspondence Address: Brown Rudnick Berlack Israels LLP, One Financial Center, Boston, MA, 02111, US

(21) Appl. No.: 11/053,286

(22) Filed: Feb. 8, 2005

(51) Int. Cl. A61F 13/15

(52) U.S. Cl. 604/385.24

(57) ABSTRACT

An absorbent article includes a unitary panel having a chassis with longitudinally opposed first and second ends and laterally opposed first and second edges. The unitary panel is separately attachable to the absorbent article, and includes first and second extensible zones located laterally distal from the first and second edges respectively of the chassis. The unitary panel includes first and second non-extensible zones located laterally distal from the first and second extensible zones. A method of making the absorbent articles includes providing a supply of panel material to an absorbent article forming station. The method also includes attaching pairs of fasteners spaced locations at sides of the material, and dividing or cutting the material to form a plurality of single-piece back panels having zoned elasticity. The method further includes attaching the back panel to an end of an article chassis, and attaching a front panel to the other end of the article chassis.

### BACKGROUND

1. Technical Field

The present disclosure generally relates to absorbent articles, and more particularly, to disposable absorbent articles that include a unitary stretch panel and a corresponding method of manufacture.

2. Background of the Related Art

Absorbent articles such as, for example, disposable diapers, training pants, adult incontinent pads, sanitary napkins, pantiliners, incontinent garments, etc. are generally worn, in cooperation with garments and disposed against a body surface by infants or adult incontinent individuals. The absorbent article is employed to collect and absorb body fluid discharge, such as, for example, blood, menses, urine, aqueous body fluids, mucus and cellular debris. For example, the absorbent article may be disposed between the legs of an individual adjacent a crotch area. The absorbent article is positioned with a garment and drawn into engagement with a body surface of the crotch area to collect body discharge.

As is known, absorbent articles typically include a fluid permeable coverstock for engaging the body surface, a fluid impermeable backsheet and an absorbent core supported therebetween. The backsheet serves as a moisture barrier to prevent fluid leakage to the garment. The absorbent core usually includes a liquid retention material that faces the body surface. The absorbent core can include, for example, loosely formed cellulosic fibers, such as, for example, wood pulp, fluff pulp, etc. for acquiring and storing body discharge. Elasticized regions can be provided around the edges of the article to improve fit and facilitate securement of the article about the waist and legs of a wearer.

Fastening of the absorbent articles with an individual can require the use of fasteners and closure tabs, including mechanical closure material, such as hook and/or loop material, adhesive or the like, that extend laterally from a front and rear section of the absorbent article. For example, a disposable diaper can be affixed to a wearer by drawing the diaper through the crotch and attaching the closure tabs that extend from a rear section across the wearer's hips to the front section of the diaper. This holds the rear and front sections of the diaper together.

In some cases, the front and/or rear sections are elasticized to facilitate attachment and improve fit. Typically, the rear section has two separate elasticized portions, one attached to each lateral side of the diaper. These types of absorbent articles, however, can result in higher production costs due to the additional material and manufacturing operations required.

It would therefore be desirable to overcome the disadvantages and drawbacks of the prior art by providing an absorbent article including a unitary stretch panel and corresponding method of manufacture.

### SUMMARY

Accordingly, an absorbent article including a unitary stretch panel and corresponding method of manufacture is disclosed. Objects and advantages of the present disclosure are set forth in part herein and in part will be obvious therefrom, or may be learned by practice of the present disclosure that is realized and attained by the instrumentalities and combinations pointed out in the appended claims for the devices and methods of the present disclosure consisting of its constituent parts, constructions, arrangements, combinations, steps and improvements herein shown and described.

In one particular embodiment, the absorbent article includes a unitary panel having a chassis with longitudinally opposed first and second ends and laterally opposed first and second edges. The unitary panel is separately attachable to the absorbent article, and includes a first extensible zone located laterally distal from the first edge of the chassis and configured to provide a conforming fit. A second extensible zone is located laterally distal from the second edge of the chassis and is configured to provide a conforming fit. A first non-extensible zone located laterally distal from the first extensible zone is designed for attachment to a first fastener. A second non-extensible zone located laterally distal from the second extensible zone is designed for attachment to a second fastener. The unitary panel further includes a central non-extensible zone located intermediate the first and second extensible zones that is configured for attachment to the first end of the chassis. The absorbent article further includes a second panel attached to the second end of the chassis.

A method of making an absorbent article, such as, for example, a diaper having a unitary panel is provided and includes providing a supply of panel material to an absorbent article forming station. The method also includes attaching pairs of fasteners at spaced locations at sides of the panel material. The panel material is cut along a plurality of panel-forming lines to form a plurality of single-piece back panels having zoned elasticity. The method further includes attaching the single-piece back panel to an end of an article chassis. A front panel is provided and attached to the other end of the article chassis.

### BRIEF DESCRIPTION OF THE DRAWINGS

The objects and features of the present disclosure are set forth with particularity in the appended claims. The present disclosure, as to its organization and manner of operation, together with further objectives and advantages may be understood by reference to the following description, taken in connection with the accompanying drawings, in which:

FIG. 1 is a plan view of an absorbent article in accordance with principles of the present disclosure;

FIG. 2 is a cutaway plan view of a manufacturing method in accordance with principles of the present disclosure;

FIG. 2A is a side view of the manufacturing method shown in FIG. 2;

FIG. 2B is an alternate view of the manufacturing method shown in FIG. 2;

FIG. 3 is a plan view of a portion of the absorbent article in FIG. 1; and

FIG. 4 is a cutaway plan view of an alternate embodiment of the manufacturing method shown in FIG. 2.

### DETAILED DESCRIPTION OF EXEMPLARY EMBODIMENTS

The exemplary embodiments of the absorbent article and methods of use disclosed are discussed in terms of absorbent articles, and more particularly, in terms of a disposable absorbent article that includes a unitary stretch panel and a corresponding method of manufacture. For example, it would be desirable if such a disposable absorbent article includes a stretch panel of single-piece construction and having zoned elasticity. The presently described absorbent article is configured to be easily manufactured in accordance with the principles of the present disclosure.

In the discussion that follows, the term “body-facing surface” refers to a portion of a structure that is oriented towards a body surface, and the “garment-facing surface” refers to a portion of the structure that is oriented towards a garment and is typically opposing the body-facing surface and may be referred to as such. As used herein, the term “body surface” refers to a portion of an individual's body that the absorbent article is disposed with for collecting and absorbing fluid discharge from the individual. As used herein, the term “absorbent article,” “absorbent garment” or “garment” refers to garments that absorb and contain body waste, and more specifically, refers to garments that are placed against or in proximity to the body of the wearer to absorb and contain the various body discharges and wastes.

A non-exhaustive list of examples of absorbent articles includes diapers, training pants, diaper covers, disposable diapers, feminine hygiene products and adult incontinence products. The term absorbent articles includes all variations of absorbent garments, including disposable absorbent garments that are intended to be discarded or partially discarded after a single use and unitary disposable absorbent garments that have essentially a single structure. As used herein, the term “training pants” refers to an absorbent article generally worn by children about the lower torso. The absorbent article may be disposable or partially discarded after a single use. The absorbent article can be a unitary structure or include replaceable inserts or interchangeable parts.

Absorbent articles and diapers may have a number of different constructions. In each of these constructions it is generally the case that an absorbent core is disposed between a liquid pervious, body-facing topsheet and a liquid impervious, exterior facing backsheet. In some cases, one or both of the topsheet and backsheet may be shaped to form a pant-like article, such as a training pant. In other cases, the topsheet, backsheet and absorbent core may be formed as a discrete assembly that is placed on a main chassis layer and the chassis layer is shaped to form a pant-like absorbent article. The absorbent article may be provided to the consumer in the fully assembled pant-like shape or may be partially pant-like and require the consumer to take the final steps necessary to form the final pant-like shape. For a diaper application, an open article is provided and wrapped about a child's waist, such as by manually fastening one or more fasteners or fastener tabs.

The following discussion includes a description of the absorbent article in accordance with the present disclosure. Reference will now be made in detail to the exemplary embodiments of the disclosure, which are illustrated in the accompanying FIGURES.

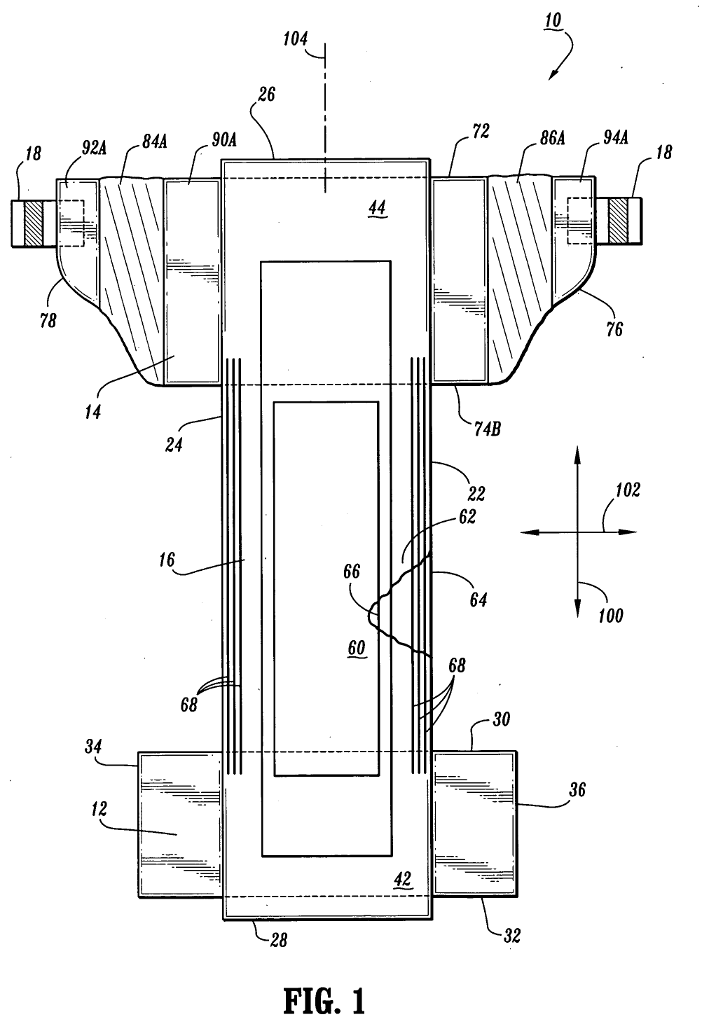

Turning now to the FIGURES, wherein like components are designated by like reference numerals throughout the several views. Referring to FIGS. 1-3, there is illustrated an absorbent article 10, constructed in accordance with the principles of the present disclosure, such as, for example, a diaper. The absorbent article 10, as shown in FIG. 1, generally has a longitudinal direction 100 that extends generally parallel to the front-to-back axis of a wearer, and a lateral direction 102 that extends generally parallel to the side-to-side axis of a wearer. The absorbent article 10 generally is symmetrical about a longitudinal centerline 104, but also may have asymmetrical components or shapes. The terms “proximal” and “distal,” as used herein, refer to positions generally along the lateral direction 102, with proximal locations being located closer to the longitudinal centerline 104 than distal locations. Those skilled in the art will also recognize that “front” and “back” (or “rear”) are relative terms, and these regions may be transposed without departing from the scope of the present invention.

The absorbent article 10 includes a front panel 12, a rear panel 14, and an intermediate section (chassis) 16 forming the crotch portion of the absorbent article. The rear section of the absorbent article 10 is in the form of a single-piece (unitary) panel 14 having a zoned stretch property, in that only certain zones thereof will stretch during use. The front panel 12 may or may not include extensible zones. Although the absorbent article 10 is illustrated and described as a diaper, the present invention can be utilized in other similar types of absorbent disposable articles, such as, for example, training pants, feminine hygiene products and adult incontinence products. It should be pointed out that as used herein the term “disposable” refers to an absorbent article designed to be used until soiled, either by body discharge or otherwise, and then discarded, rather than being washed and used again.

The unitary back panel 14 has a linear rear edge 72, a linear front edge 74B, and an opposed pair of side edges 76, 78 that are contoured to enhance fit, comfort, aesthetics and performance. The panel 14 includes a first non-extensible zone, such as, for example, a left non-stretch zone 92A configured for attachment to the closure tab 18. A second non-extensible zone, such as, for example, a right non-stretch zone 94A is configured for attachment to the other closure tab 18. A third non-extensible zone, such as, for example, a central non-stretch zone 90A is configured for attachment to the article chassis 16. A first extensible zone, such as, for example, a left stretch zone 84A is intermediate the left non-stretch zone 92A and the central non-stretch zone 90A. The left stretch zone 84A is configured to stretch in the lateral direction 102. A second extensible zone, such as, for example, a right stretch zone 86A is intermediate the right non-stretch zone 94A and the central non-stretch zone 90A. The right stretch zone 86A is configured to stretch in the lateral direction 102.

The unitary panel 14 is disposed with first end 44 of the article chassis 16 such that the left stretch zone 84A is positioned laterally distal from the left edge 24 of the chassis 16 and the right stretch zone 86A is positioned laterally distal from the right edge 22 of the chassis 16.

The front panel 12 can be non-extensible and includes a linear rear edge 30, a linear front edge 32, and an opposed pair of linear side edges 34, 36. The chassis 16 forming the crotch portion of the absorbent article 10 is an elongated member that includes an opposed pair of end regions 42, 44, which are fixedly secured to the inner surface of the front and back panels 12, 14, respectively. The chassis 16 is a planar member that is elasticized to form a cup-shaped or bucket-shaped member to conform to the crotch of the wearer between the wearer's legs. The chassis 16 includes a pair of longitudinal sides 22, 24, and a pair of shorter linear end edges 26, 28.

The left edge 78 of the back panel 14 is arranged to be releasably secured by at least one fastening tape 18, e.g., an adhesive tape or a multi-hook fastening tape, to a portion of the front panel 12 adjacent the side edge 34 thereof. In a similar manner the right edge 76 of the back panel 14 is arranged to be releasably secured by at least one similar fastening tape 18 to a portion of the front panel 12 adjacent its side edge 36. Depending upon the material(s) forming the front panel 12 and the construction of the fastening tapes 18, the front panel includes “landing zones” (not shown). The landing zones provide areas on the front panel to which the fastening tapes 18 are be releasably secured to enable the wearer to achieve a good fit and to enable the absorbent article brief to be put on and taken off repeatedly.

The article chassis 16 may include, for example, a moisture-pervious cover-stock layer 62 and a moisture impervious barrier layer 64 which are each of rectangular shape and of the same size. The cover-stock layer 62 can be of any suitable construction, such as a conventional non-woven material. One particularly suitable material is spunbonded polypropylene (SBPP) non-woven, such as sold under the trade designation 15 gsm polypropylene non-woven by Avgol of Holon, Israel. The barrier layer 64 can also be of any suitable construction, e.g., a conventional polyethylene film or a conventional poly-laminate (PLAM). One particularly suitable film is that sold under the trade designation 1.0 mil cast polyethylene XC3-222-1491.1 by Pliant Corporation of Williamsburg, Va. One particularly suitable PLAM is that sold under the trade designation XLAM #8B4535 by Pliant Corporation of Williamsburg, Va. A conventional fluid absorbent core 60 of generally rectangular shape is interposed between the cover-stock layer 62 and the moisture barrier layer 64. The width of the core 60 is less than that of the cover-stock layer 62 and moisture barrier layer 64 so that the longitudinally extending sides 22, 24 of the chassis 16 are extremely flexible and conformable to the anatomy of the wearer, as will be described later.

The article chassis 16 is elasticized along its marginal edges 22, 24 to enable the chassis 16 when worn to form a cup-shaped or bucket shaped configuration having a pair of upstanding barrier walls. To that end, plural elongated pre-tensioned threads or strands 68 of any suitable elastic material are adhesively secured between the marginal edges of the cover-stock layer 62 and the barrier layer 64. The strands 68 can be of any suitable elastic material. One such material is Lycra Elastics sold under the trade designation of Decitex 740 by E. I. DuPont DeNemours and Company, Inc. of Wilmington, Del.

The core 60 is of any suitable construction such as a fluff, e.g., comminuted wood pulp or other cellulosic fibers. Enhanced absorbency for the core can be accomplished by use of an air-laid super absorbent material, or by the inclusion of absorbency enhancers, for example, materials sometimes referred to as “super-absorbent-polymers” (which may be in the form of particles or fibers). Examples of super absorbent materials are hydrogel polymer particulates, sometimes referred to as “SAP,” and hydrogel polymer fibers, sometimes referred to as “SAF.” The fluff for the core 60 serves as a means for quickly absorbing the liquid transferred to it. Unfortunately, while fluff is suitable for the task of quick absorption, it is somewhat deficient from the standpoint of liquid retention and ability to accommodate repeated insults. SAP on the other hand, has the ability to retain absorbed liquid and to handle repeated insults. The drawback with SAP and SAF are their slowness in absorbing liquid. Accordingly, the chassis 16 may also include an optional fluid acquisition layer 66 disposed over the core 60. The acquisition layer 66 can be of any suitable material such as a woven, non-woven or carded fibrous material. One particularly suitable material is TABICO FF, sold by Polymer Group, Inc. of Charleston, S.C. The acquisition layer 66 serves to quickly absorb the liquid through the cover stock for temporary retention, and to transfer that liquid into the underlying core 60 at a rate at which the core can absorb for final or permanent retention. The acquisition layer 66 may be in the form of a rectangular strip of slightly less width than the core 60 and which extends for a substantial length of the core 60 centered over it in the area which will receive the urine/feces insult.

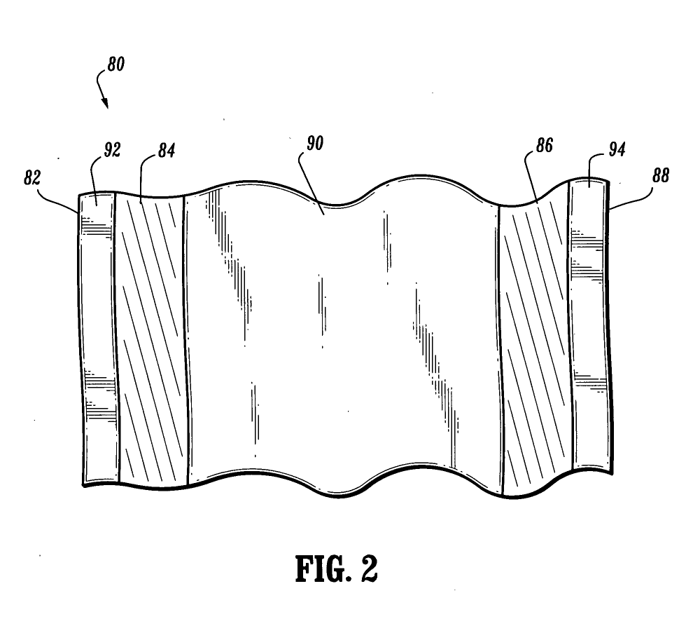

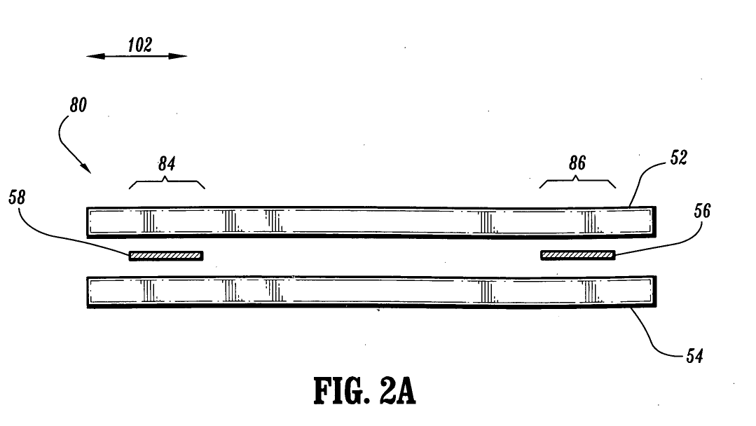

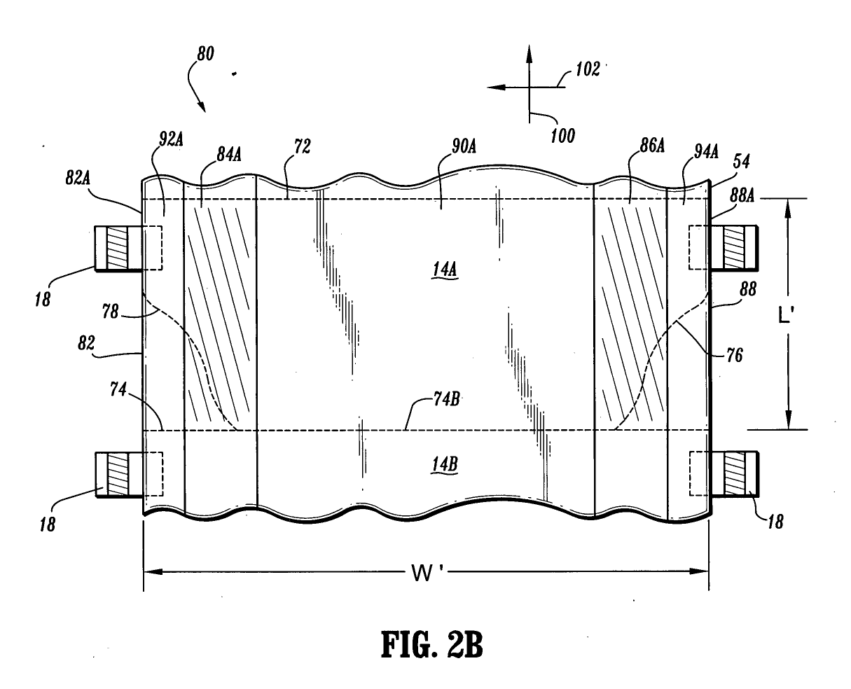

The present disclosure also includes a method of making disposable articles, such as, for example, absorbent article 10 described with regard to FIG. 1, including the unitary back panel 14 having zoned elasticity. The method includes providing a supply of panel material 80, as shown in FIGS. 2, 2A and 2B, to an absorbent article forming station. The material 80 includes a right edge 88, a left edge 82, spaced apart extensible zones 84, 86, and non-extensible zones 90, 92, 94.

The panel material 80 from which the panels 14 are to be formed can be a composite that includes layers, as shown in FIG. 2A, of elastic material and non-elastic material. The material 80 includes a first non-elastic substrate, such as, for example, a first non-woven layer 52. The material 80 also includes a second non-elastic substrate, such as, for example, a second non-woven layer 54. Laterally spaced apart elastic materials, such as, for example, a first elastic material 56 and a second elastic material 58, are sandwiched between the non-woven layers 52, 54. The non-woven layers 52, 54 and elastic materials 56, 58 are joined together to form a substantially unitary structure or panel material 80.

To fabricate the panel material 80, the elastic materials 56, 58 are aligned in the extensible zones 84, 86 and adhered to the non-woven layers 52, 54 under “stretched conditions.” The stretch is executed in the cross (lateral) direction 102 by releasing the tension in the elastic materials 56, 58. This contracts and draws the non-wovens 52, 54 along extensible zones 84, 86 into crosswise pleats (i.e., shirrs, corrugations) of a desired thickness and frequency.

Suitable elastic material 56, 58 in the form of strands, threads, film or the like may be used. Various commercially available materials may be used, such as natural rubber, butyl rubber or other synthetic rubber, urethane, elastomeric materials such as spandex, which is marketed under various names, including LYCRA® (DuPont), GLOSPAN™ (Globe) and SYSTEM 7000# (Fulflex). The present invention is not limited to any particular elastic material or to any particular shape, size or number of elastics.

The non-woven layers 52, 54 may be a conventional spunbond-meltblown-spunbond (SMS) material sold under the trade designation polypropylene non-woven by Avgol of Holon, Israel. The SMS material may be of any suitable weight, for example, 13.5 grams per square meter (gsm), and any suitable thickness, for example, 0.6 mil.

The bonding between the layers of the panel material 80 may be accomplished using joining methods known in the art, such as, for example, compression bonds, heat bonds, ultrasonic bonds, adhesives and the like, or combinations of different bonding methods. The material 80 can be made directly in-line, or manufactured off-line and wound up on the roll.

The panel material 80 is brought to a station where pairs of opposing fasteners 18, as shown in FIG. 2B, are registered and attached at spaced locations to respective sides of the material 80. The panel material 80 is cut, for example, using a die cutter to form a repeating pattern of identically shaped and like-oriented panels 14. The material 80 can be cut along a plurality of panel-forming lines to produce a plurality of single-piece panels 14 each having a zoned stretch property in that only certain zones thereof will stretch during use. Panel 14 includes spaced apart stretch zones 84A, 86A and spaced apart non-stretch zones 92A, 90A, 94A which are arranged correspondingly to arrangement of extensible and non-extensible zones 84, 86, 92, 90, 94 of the panel material 80 (FIGS. 2 and 2A). By cutting the panel material 80 in this way, waste of the material 80 between and surrounding the panels 14 is greatly reduced.

Panel-forming cuts are made in the panel material 80 along a plurality of lateral lines. The lateral cuts (lines) each extend the width W′ of the material 80 and are spaced longitudinally a distance L′ from each other. For example, a first line 72 extends between the right and left edges 82, 88 of the material 80. A second line 74 is disposed forward from the first line 72 and runs between the left and right edges 82, 88. Similar lateral cuts (not shown) are made in a repeating pattern along the length of the material 80.

Other panel-forming cuts are made in the panel material 80 along a plurality of contoured lines. For example, a third line 78 extends in a contoured manner between the left edge 82 and a point on line 74 to the right of edge 82. A fourth line 76 extends in a contoured manner between the right edge 88 and a point on line 74 to the left of edge 82. Similar curved lines (not shown) are made in a repeating pattern along the material 80.

Cutting the panel material 80 forms single-piece panels, such as, for example, a first panel 14A and a second panel 14B (shown partially) adjacent and forward thereof, with minimal waste of the material 80. For purposes of example, the panel 14 can be oriented for attachment to the rear end 44 of the chassis 16 shown in FIG. 1. The linear line 72 cut in the panel material 80 delineates the rear edge 72 of the panel 14A. A portion of the line 74 delineates the front edge 74b of the panel 14A. The line 74 also defines the rear edge of the second panel 14B. The line 76 defines a curved right edge of the panel 14A. The line 78 defines a curved left edge of the panel 14A. A left linear edge 82A of the panel 14A is defined by a portion of the panel material edge 82. A right linear edge 88A of the panel 14A is defined by a portion of the panel material edge 88.

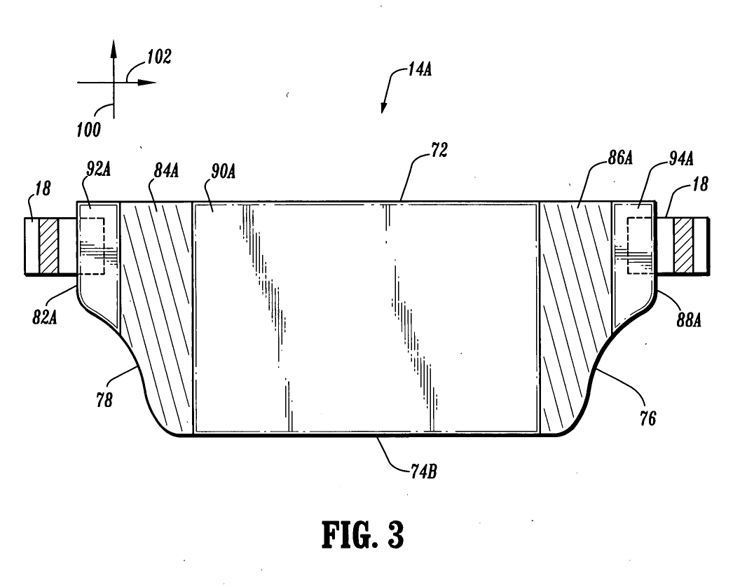

The single-piece panels 14 formed from the panel material 80 each include, as shown in FIG. 3, a first non-extensible zone, such as, for example, a left non-stretch zone 92A configured for attachment to the closure tab 18. A second non-extensible zone, such as, for example, a right non-stretch zone 94A is configured for attachment to the other closure tab 18. A third non-extensible zone, such as, for example, a central non-stretch zone 90A is configured for attachment to the chassis 16 of FIG. 1. A first extensible zone, such as, for example, a left stretch zone 84A is intermediate the left non-stretch zone 92A and the central non-stretch zone 90A. The left stretch zone 84A is configured to stretch in the lateral direction 102. A second extensible zone, such as, for example, a right stretch zone 86A is intermediate the right non-stretch zone 94A and the central non-stretch zone 90A. The right stretch zone 86A is configured to stretch in the lateral direction 102.

The unitary panel 14 is attached along its central non-stretch zone 90A to the first end 44 of the chassis 16 (FIG. 1) at the absorbent article forming station. In this way, the left stretch zone 84A of the panel 14 is positioned laterally distal from the left edge 24 of the chassis 16, and the right stretch zone 86A is positioned laterally distal from the right edge 22 of the chassis 16. Attachment of the panel 14 to the chassis 16 may be by one or a combination of adhesive, ultrasonic, thermal bonding or the like. Panel 14 may be attached to various types of absorbent articles.

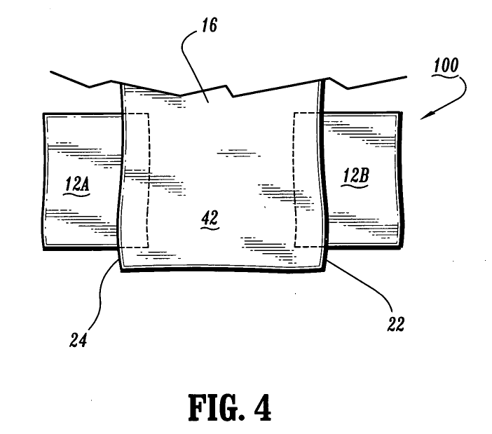

A front panel 12 (FIG. 1), is cut (not shown) and placed on the front end 42 of the chassis 16 at the absorbent article forming station. The front panel 12 can be secured to the chassis 16 by one of adhesive, ultrasonic, thermal bonding, etc. The front panel 12 may be, for example, rectangular, and of single-piece construction. However, it is envisioned that the front panel 12 can be constructed of various materials in a variety of configurations and arrangements. In an alternate embodiment of the article 10, as shown in FIG. 4, the front panel is comprised of two separate portions 12A, 12B one disposed on each side of the chassis 16. The first portion 12A of the panel is attached to the left edge 24 at the front end 42 of the chassis 16. The second portion 12B is attached to the right edge 22 at front end 42. Attachment of the first and second portions 12A, 12B to the chassis 16 may be by one or a combination of adhesive, ultrasonic, thermal bonding or the like. The portions 12A, 12B can be made from a variety of inelastic materials, such as, for example, non-woven materials. Such non-woven material can be a conventional spunbond-meltblown-spunbond (SMS) material sold under the trade designation polypropylene non-woven by Avgol of Holon, Israel. The SMS material may be of any suitable weight, for example, 27.0 grams per square meter (gsm), and any suitable thickness, for example, 1.2 mil.

In an alternative embodiment, the article chassis 16 can also include a single-piece panel 14 and fasteners 18, without the fist panel 12. In this embodiment, the first end 42 of the chassis 16 would include a land zone or zones for fasteners 18 to attach during product use.

As described above, the method of the present disclosure provides the various components, that is, the chassis 16, the single-piece panel 14 having zoned elasticity, the front panel 12, and the fasteners 18, and combines these components to economically make an absorbent article with reduced waste of materials and decreased production resources.

The claims are intended to cover all of the foregoing classes of absorbent articles, without limitation, whether disposable, unitary or otherwise. These classifications are used interchangeably throughout the specification, but are not intended to limit the claimed invention. The invention will be understood to encompass, without limitation, all classes of absorbent articles, including those described above.

The invention of the present disclosure may be embodied in other specific forms without departing from the spirit or essential characteristics thereof. The present embodiments are therefore to be considered in all respects as illustrative and not restrictive, the scope of the invention being indicated by the appended claims rather than by the foregoing description, and all changes which come within the meaning and range of equivalency of the claims are therefore intended to be embraced therein.

What is claimed is:

1. An absorbent article comprising:
a chassis with longitudinally opposed first and second ends and laterally opposed first and second edges;
a unitary panel separately attachable to the absorbent article and including:
a first extensible zone located laterally distal from the first edge of the chassis and being configured to provide a conforming fit,
a second extensible zone located laterally distal from the second edge of the chassis and being configured to provide a conforming fit,
a first non-extensible zone located laterally distal from the first extensible zone and designed for attachment to a first fastener,
a second non-extensible zone located laterally distal from the second extensible zone and designed for attachment to a second fastener, and
a central non-extensible zone located intermediate the first and second extensible zones and configured for attachment to the first end of the chassis.

2. The absorbent article recited in claim 1, further comprising a second panel attached to the second end of the chassis.

3. The absorbent article recited in claim 1, wherein the unitary panel includes a plurality of layers.

4. The absorbent article recited in claim 1, wherein the unitary panel includes a first non-elastic substrate and a second non-elastic substrate, such that a first elastic material and a second elastic material are disposed between the first and the second non-elastic substrates.

5. The absorbent article recited in claim 4, wherein the first and second elastic materials are aligned respectively in the first and second extensible zones and adhered to the first and second non-elastic substrates using adhesives.

6. The absorbent article recited in claim 5, wherein the first and the second elastic materials are configured to contract the first and the second non-elastic substrates along the extensible zones into pleats.

7. The absorbent article recited in claim 2, wherein the second panel is non-extensible.

8. The absorbent article recited in claim 7, wherein the second panel includes landing zones for engaging fasteners attached to the first and second non-extensible zones.

9. A method of making an absorbent article, the method comprising the steps of:
providing a panel material;
attaching fasteners to the panel material;
cutting the panel material to form a plurality of identically shaped unitary panels each having two spaced apart extensible zones;
providing a chassis having longitudinally opposed first and second ends and laterally opposed first and second edges; and
attaching the unitary panel to the first end of the chassis such that a first and a second extensible zones are positioned distal from the first and second chassis edges respectively.

10. The method recited in claim 9, further comprising the step of providing a second panel and attaching it to the second end of the chassis.

11. The method recited in claim 9, wherein the unitary panel further includes two spaced apart non-extensible zones.

12. The method recited in claim 11, wherein a first non-extensible zone is located laterally distal from the first extensible zone.

13. The method recited in claim 12, wherein the first non-extensible zone is configured for attachment to a first fastener.

14. The method recited in claim 11, wherein non-extensible zones of the panel comprise non-woven material.

15. The method recited in claim 9, wherein the extensible zones are configured to extend only in a lateral direction.

16. The method recited in claim 9, wherein the unitary panel further includes a central non-extensible zone configured for attachment to the chassis.

17. A method of making an absorbent article, the method comprising the steps of:
providing a panel material having first and second lateral sides;
attaching pairs of opposing fasteners to the panel material at the lateral sides thereof;
cutting the panel material to form a plurality of unitary panels each including:
a first extensible zone spaced apart from a second extensible zone,
a first non-extensible zone spaced apart from a second non-extensible zones,
and a central non-extensible zone intermediate the first and second extensible zones;
providing a rectangular chassis having an absorbent core, longitudinally opposed first and second ends, and laterally opposed first and second edges; and
attaching the unitary panel, along its central non-extensible zone, to the first end of the chassis such that the first extensible zone is disposed laterally distal from the first edge of the chassis and the second extensible zone is disposed laterally distal from the second edge of the chassis.

18. The method recited in claim 17, further comprising the step of providing a rectangular second panel and attaching it to the second end of the chassis.

19. The method recited in claim 17, wherein the panel material is die cut to form unitary panels each having a top linear edge, a bottom linear edge, a first curved lateral edge and a second curved lateral edge.

20. The method recited in claim 17, wherein a cut made laterally in the panel material defines a bottom linear edge of a first unitary panel and also defines a top linear edge of a second unitary panel.

21. The method recited in claim 17, wherein an edge portion of the panel material defines an edge of the unitary panel configured for attachment to a fastener.

22. The method recited in claim 17, wherein contoured cuts made in the material traverse the extensible zones.

23. The method recited in claim 17, wherein the extensible zones are configured to provide a conforming fit.
